(19) United States
(12) Patent Application Publication
(10) Pub. No.: US 2016/0331876 A1
(43) Pub. Date: Nov. 17, 2016

(54) FLUID COLLECTION AND DISPOSAL SYSTEM

(71) Applicants: Larry C. Smith, Shawnee, KS (US); Christopher Fedele, Mentor, OH (US)

(72) Inventors: Larry C. Smith, Shawnee, KS (US); Christopher Fedele, Mentor, OH (US)

(21) Appl. No.: 15/152,915

(22) Filed: May 12, 2016

Related U.S. Application Data
(60) Provisional application No. 62/160,958, filed on May 13, 2015.
(60) Provisional application No. 62/189,694, filed on Jul. 7, 2015.

(51) Int. Cl. A61M 1/00; A61B 50/13

(52) U.S. Cl. CPC A61M 1/0025; A61M 1/0031; A61B 50/13; A61M 2205/18; A61M 2205/3313; A61M 2230/20; A61M 2230/005; A61M 2205/52; A61M 2205/502

(57) ABSTRACT

A medical waste fluid collection cart may include one or more sensors, a controller in communication with at least one of the sensors, and a memory coupled to the controller, the memory configured to store one or more algorithms, measurement correlations, and/or look-up tables relating sensed measurements to concentrations of waste fluid received from a surgical site. The fluid collection cart may further include a container and a suction tube having a lumen extending through the suction tube, where the suction tube may communicate with the container to deposit waste fluid from a surgical site in the container. One or more of the sensors may include a light sensor for sensing a measure related to a concentration of blood in the waste fluid removed from a surgical site. The controller may determine a blood loss quantity from identifying an average blood concentration in the waste fluid removed from the surgical site.

### CLAIM OF PRIORITY

This application claims the benefit of U.S. Provisional Patent Application Ser. No. 62/160,958, filed on May 13, 2015, and also claims the benefit of U.S. Provisional Patent Application Ser. No. 62/189,694, filed on Jul. 7, 2015, the benefit of priority of each of which is claimed hereby, and each of which are incorporated by reference herein in its entirety.

### TECHNICAL FIELD

The present disclosure relates generally to systems and methods for collecting fluids during medical procedures and, in particular, to sensing systems for use in determining blood loss from a patient at a surgical site.

### BACKGROUND

Biological fluids and other types of medical waste often must be collected during surgery or other medical procedures. This is typically accomplished using a medical waste fluid collection cart, which may be part of a medical waste fluid collection and disposal system. Such carts may include at least one suction canister where a vacuum port on the canister lid is connected to a source of vacuum via a hose or line. As a result, a vacuum is drawn on the interior of the canister. A second hose or line is connected to a “patient” suction port on the canister lid and is used to collect medical waste in the form of fluids and solids from the patient, which is stored in the canister. After collection, the medical waste and contaminated collection components, such as canister lids, and the like, must be disposed of in accordance with rules and regulations imposed by various government and regulatory organizations.

### SUMMARY

The disclosure is directed to several alternative or complementary designs for, materials for, and methods of using medical device structures, systems, and assemblies. Although it is noted that conventional medical waste fluid collection systems exist, there exists a need for improvement on those devices.

Accordingly, a fluid collection system may include a container, suction tubing or a suction line having a lumen extending at least partially through the suction tubing, and a light sensor. Illustratively, the suction tubing may be in communication with the container to deposit waste fluid removed from a surgical site and passing through the lumen of the suction tubing into the container. The sensor may be configured to sense a measure related to a concentration of blood in the waste fluid removed from the surgical site.

In some instances, the sensor of the fluid collection system may include a light source and a light detector. The light detector may be configured to detect light from the light source to sense a measure related to a concentration of blood in waste fluid removed from a surgical site.

Alternatively, or in addition, the fluid collection system may include a memory and a processor in communication with the memory and the light sensor. The memory may be configured to store correlations between the sensed measure related to blood concentration and blood concentration in waste fluid. The processor may be configured to estimate an average blood concentration of the removed waste fluid over a period of time based at least in part on sensed measures related to blood concentration and the stored correlations between the sensed measures related to blood concentration and blood concentration in the waste fluid received from a surgical site.

In operation, the fluid collection system may be used in a method of determining blood loss from a patient during a surgical procedure on the patient. The method may include receiving waste fluid from a surgical site through, for example, a fluid collection system. Then, sensing a measure related to light after the light has interacted with the waste fluid received from the surgical site as the waste fluid is received at the fluid collection system. From the measure related to light that was sensed after the light interacted with the waste fluid received from the surgical site, a measure related to blood concentration of the fluid received from the surgical site may be determined.

The above summary of some example aspects is not intended to describe each disclosed embodiment or every implementation of the claimed disclosure.

### BRIEF DESCRIPTION OF THE DRAWINGS

The disclosure may be more completely understood in consideration of the following detailed description of various embodiments in connection with the accompanying drawings, in which:

FIG. 1 is a schematic perspective view of a medical waste fluid collection and disposal system including a medical waste fluid collection cart;

FIG. 2 is a schematic perspective view of a lid of a medical waste fluid collection cart;

FIG. 3 is a schematic block diagram representation of an illustrative controller for a medical waste fluid collection cart;

FIG. 4 is a schematic cross-sectional view taken along a sensor positioned about suction tubing of a medical waste fluid collection and disposal system;

FIG. 5 is schematic cross-sectional view taken along a sensor positioned about suction tubing of a medical waste fluid collection and disposal system;

FIG. 6 is a schematic cross-sectional view taken along a sensor positioned in suction tubing a of a medical waste fluid collection and disposal system;

FIG. 7 is a schematic cross-sectional view taken along a sensor positioned in suction tubing of a medical waste fluid collection and disposal system;

FIG. 8 is a schematic perspective view of a lid of a medical waste fluid collection cart with an auxiliary chamber;

FIG. 9 is a schematic cross-sectional view of an auxiliary chamber with a schematic side view of suction tubing and a suction port;

FIG. 10 is a schematic cross-sectional view of an auxiliary chamber with a schematic side view of suction tubing and a suction port;

FIG. 11 is a schematic side view of a medical waste fluid collection cart;

FIG. 12 is a schematic side view of a medical waste fluid collection cart; and

FIG. 13 is a schematic flow diagram of an illustrative method of operating a medical waste fluid collection cart.

While the disclosure is amenable to various modifications and alternative forms, specifics thereof have been shown by way of example in the drawings and will be described in detail. It should be understood, however, that the intention is not to limit aspects of the claimed disclosure to the particular embodiments described. On the contrary, the intention is to cover all modifications, equivalents, and alternatives falling within the spirit and scope of the claimed disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about”, when referring to numeric values, generally refers to a range of numbers that one of skill in the art would consider equivalent to the recited value (i.e., having the same function or result). In many instances, the term “about” may be indicative as including numbers that are rounded to the nearest significant figure. Additionally, or alternatively, the term “about” may generally refer to the area around an object or to a first object positioned at least partially around a second object.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the claimed disclosure. The illustrative embodiments depicted are intended only as exemplary. Selected features of any illustrative embodiment may be incorporated into an additional embodiment unless clearly stated to the contrary.

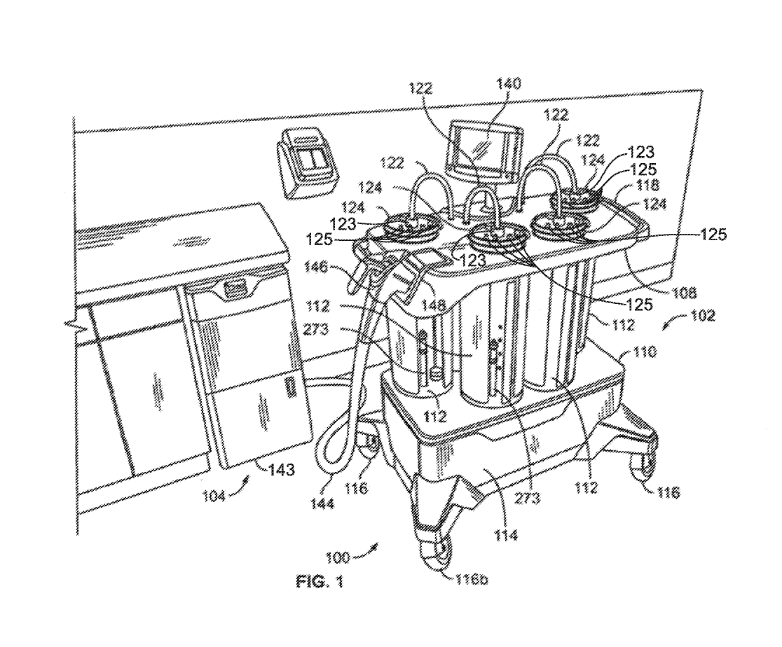

FIG. 1 is a perspective view of a medical waste fluid collection and disposal system 100 including a medical waste fluid collection cart 102 and an evacuation station 104. An illustrative medical waste fluid collection and disposal system, which is not meant to be limiting in any way, is disclosed in, for example: U.S. Pat. No. 8,292,857 filed on Oct. 6, 2008, entitled “MEDICAL WASTE FLUID COLLECTION AND DISPOSAL SYSTEM”, the entirety of which is incorporated herein by reference for all purposes. In some cases the medical waste fluid collection cart 102 may include a top plate 108 and a bottom plate 110. Positioned between the top and bottom plates are one or more fluid collection canisters or containers 112 (e.g., cylinders or other canisters or containers). While four containers are shown in FIG. 1, the medical waste fluid collection cart 102 may include an alternative number of containers, for example, the medical waste fluid collection cart 102 may be configured to include one container, two containers, or another number of containers, if desired. The medical waste fluid collection cart 102 also may include a bottom cabinet 114 that may be mounted on casters 116. The casters 116 may be used to provide steering capability for the cart 102. The casters 116 may include a brake feature. The top plate 108 features a handle 118. As a result, the medical waste fluid collection cart 102 may be easily pushed to and from an operating room.

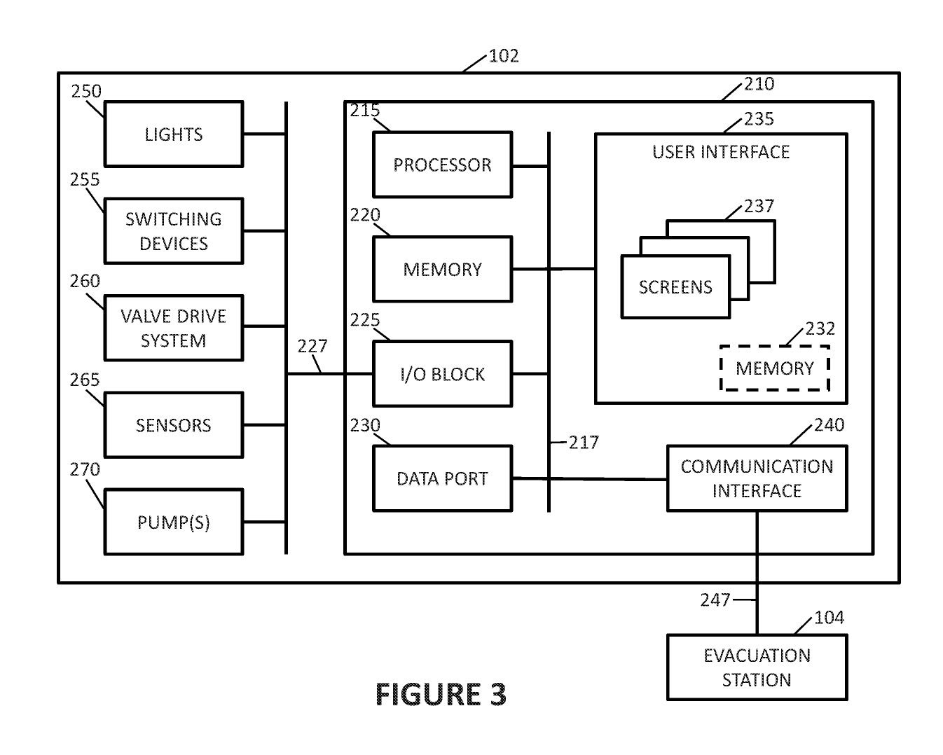

In some cases, each of the containers 112 may be connected between a number of corresponding lids 124 or manifolds (e.g., a manifold that may be connected to a lid of a container or providing fluid access to a container) via flexible tubing 122 to a vacuum source. The lids or manifolds may be constructed of molded plastic or other material and may be disposable. The flexible tubing 122 of FIG. 1 may attach at one end to a vacuum port of the lid 124 or manifold, with the opposite end attaching to a vacuum pump. The vacuum pump may be provided on the cart 102, or the vacuum tubing may be connected to a vacuum source provided in the medical facility, such as a vacuum pump associated with wall suction provided in an operating room. The vacuum port may be provided with a filter, such as a hydrophobic filter, positioned in a compartment in the lid 124 or in a manifold. A patient suction tube (e.g., a reusable or disposable suction tube) may be connected to a suction port on the lid 124 or the manifold, while one or more additional ports may be covered by caps or covers. The lid 124 or manifold also may include a tubing post for occluded vacuum with the patient tubing. As a result, vacuum or suction is selectively drawn on each container so that fluids may be collected in containers during the medical procedure via the suction tubing extending from the container to the patient. The suction and other functions of the medical waste fluid collection cart 102 may be controlled via a user interface 140 (e.g., a touch screen), which may include and/or may be communicably coupled to the controller of FIG. 3.

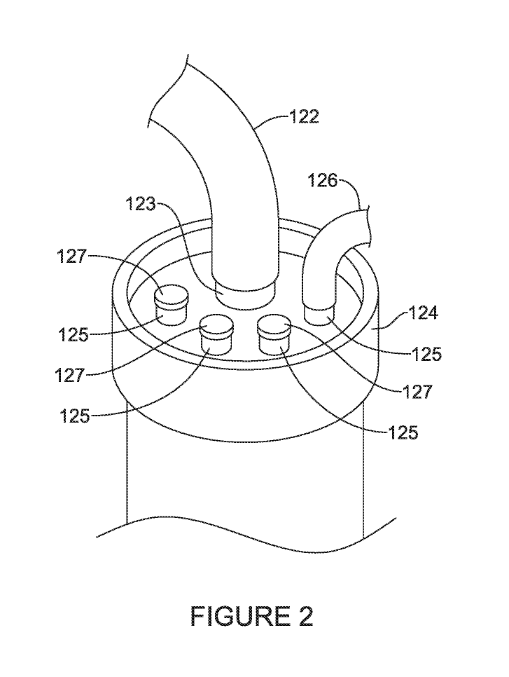

FIG. 2 is an illustrative depiction of a lid 124 or manifold with flexible tubing 122 connected to a vacuum port 123 and a suction line or tube 126 connected to a suction port 125, where each of the flexible tubing 122 and the suction line or tube 126 may include a lumen extending at least partially therethrough. In some cases, suction ports 125 to which a suction tube 126 is not attached, if any, may be covered with a cover or cap 127. In operation, a vacuum source may create a negative pressure within the container 112 to pass fluid removed from a surgical site through the lumen of the suction tube 126 and deposit the removed fluid in a container 112.

After the medical waste fluid collection cart 102 is used in an operating room procedure, and fluids have been collected in one or more of the containers 112, the patient suction tubing 126 is removed from the container lids 124 and all suction ports 125 for each container lid 124 to which suction was applied are covered with covers or caps 127. The medical waste fluid collection cart 102 may then be rolled to a position adjacent to the evacuation station 104 for draining, washing, disinfecting, rinsing and return to a suction collection state, as illustrated in FIG. 1.

The evacuation station 104 may include a housing 143 that contains a source of disinfection solution, pump, drainage system, and/or other components for draining, washing, and disinfecting the containers of the medical waste fluid collection cart 102. The evacuation station 104 communicates with the medical waste fluid collection cart 102 by way of the composite hose 144 and the coupler 146. As illustrated in FIG. 1, the coupler 146 is received within a receptacle 148 of the medical waste fluid collection cart 102. Washing fluid from the evacuation station 104 may be dispensed in the containers 112 with cleaning nozzles 273 positioned in the containers 112 to wash and disinfect the containers 112.

When the draining, washing and disinfecting of the cart containers 112 is complete, the coupler 146 is removed from the receptacle 148 on the medical waste fluid collection cart 102 so that the medical waste fluid collection cart 102 may again be rolled to an operating room for use. The coupler 146 permits a single connection to be made between the medical waste fluid collection cart 102 and the evacuation station 104. This simplifies and expedites connecting the medical waste fluid collection cart 102 to, and disconnecting the medical waste fluid collection cart 102 from, the evacuation station 104. When the medical waste fluid collection cart 102 and the evacuation station 104 are connected using the coupler 146, one or more communication links may be used to permit a controller of the medical waste fluid collection cart 102 and a controller of the evacuation station 104 to communicate so that the user can control both, including one or more of a draining operation, a washing operation and a disinfecting operation, via the touch screen 140.

FIG. 3 is a block diagram representation of an illustrative controller 210 for the medical waste fluid collection cart 102 of FIG. 1. In some cases, the medical waste fluid collection cart 102 may include one or more components capable of processing instructions for controlling one or more functions of the medical waste fluid collection cart 102. In some cases, the controller 210 may be included within the user interface 140 of FIG. 1. Alternatively, or in addition, one or more components of the controller 210 may be provided and/or mounted on the medical waste fluid collection cart 102 separate from the user interface 140. The controller 210 may include a processor 215 (e.g. microprocessor, microcontroller, etc.) that may be communicatively coupled via a data bus 217 to one or more components of the controller 210 including one or more units of memory 220, 232 (or memory located remote from the controller 210), an input/output block 225, a data port 230, a user interface 235, and/or a communication interface 240. In some cases, the user interface 235 may be configured to display one or more screens 237 to a user.

The processor 215 may operate using a control algorithm that controls or at least partially controls the collection of medical waste fluid and/or disposal of the collected medical waste fluid. The processor 215 may, for example, operate in accordance with an algorithm for collecting medical waste fluid from a surgical site during a medical procedure using one or more vacuum levels and/or fluid flow rates that may be specified by a user in real time and/or preset in the memory 220, 232, or other memory, for example.

In one example, the processor 215 may be configured to operate the algorithm(s) using an operating system (e.g., Windows, OS X, iOS, Android, Linux, Unix, GNU, etc.), or an example embedded operating system (e.g., QNX, NiagaraAX, Windows CE, etc.). In some cases, the controller 210 may include a timer (not shown). The timer may be integral to the processor 215 or may be provided as a separate component.

The memory 220, 232 of the illustrative controller 210 may be communicatively coupled to the processor 215. The memory 220, 232 may be used to store any desired information, such as the aforementioned control algorithm, a power monitoring algorithm, the configuration of the medical waste fluid collection and disposal system 100, set points, vacuum levels, flow levels, flags, indicators, diagnostic limits, look-up tables, sensed parameter correlations, and/or the like. The memory 220, 232 may be any suitable type of storage device including, but not limited to, RAM, ROM, EPROM, flash memory, a hard drive, and/or the like. In some cases, the processor 215 may store information within memory 220, 232, and may subsequently retrieve the stored information.

In some cases, the controller 210 may include an input/output block (I/O block) 225 for receiving one or more signals from one or more components of the medical waste fluid collection cart 102 and/or for providing one or more signals to the one or more components of the medical waste fluid collection cart 102. For example, the I/O block 225 may be configured to provide signals to and/or to receive signals from one or more lights 250 (e.g., container lights associated with the containers 112), one or more switching devices 255 (e.g., solenoids, relays, transistors, etc.), one or more components of a valve drive system 260 (e.g., motors, valves, etc.) including one or more components of the “wash” valve, the “drain” valve and/or the “vent” valve, one or more sensors 265 (e.g., light sensors, pressure sensors, level sensors, flow sensors, etc.), pumps 270 (e.g., a vacuum pump, an evacuation pump, etc.), and/or one or more other components via one or more communication paths 227. The I/O block 225 may be configured for wired communication via one or more terminal screws, for example, and/or wireless communication via a wireless communication interface, for example. In some cases, the I/O block 225 may be used to communicate with other sensors and/or other devices associated with a particular medical process.

In some cases, as illustrated in FIG. 3, the controller 210 may include a data port 230. The data port 230 may be a wireless port for wireless protocols such as Bluetooth™, WiFi, Zigbee, or any other wireless protocol. In other cases, data port 230 may be a wired port such as a serial port, an ARCNET port, a parallel port, a CATS port, a USB (universal serial bus) port, and/or the like. In some cases, the data port 230 may use one or more communication protocols, such as Ethernet, BACNet, LONtalk, etc., that may be used via a wired network or a wireless network. In some instances, the data port 230 may be a USB port and may be used to download and/or upload information from a USB flash drive or some other data source. Other remote devices may also be employed, as desired.

The data port 230 may be configured to communicate with the processor 215 and may, if desired, be used to upload information to the processor 215 and/or download information from the processor 215. Information that can be uploaded and/or downloaded may include, for example, values of operating parameters (e.g., vacuum levels, flow rates, volume levels, fluid concentration levels, etc.), measurement correlations, look-up tables, and/or algorithms. In some instances, the data port 230 may be used to upload previously-created configurations and/or software updates into the controller 210 to hasten the programming process.

In some cases, the data port 230 may be used to download data stored within the memory 220, 232 for analysis and/or transfer to another device. For example, the data port 230 may be used to download one or more stored flow rates and/or vacuum levels, faults and/or alerts log, sensed data, and/or calculations based on sensed data to a remote device such as a USB memory stick (also sometimes referred to as a thumb drive or jump drive), personal computer, laptop, iPAD® or other tablet computer, PDA, smart phone, or other device, as desired. In some cases, the data may be convertible to an MS EXCEL®, MS WORD®, text, XML, and/or Adobe PDF® file, if desired.

In the illustrative embodiment of FIG. 3, the user interface 235 may be any suitable user interface that permits the controller 210 to display and/or solicit information, as well as accept one or more user interactions with the controller 210. For example, the user interface 235 may permit a user to enter data such as vacuum levels, patient information, start/end times of procedures, flow rates, medical application types, and the like. In some cases, the user interface 235 may include a display and a distinct keypad. A display may be any suitable display. In some instances, the display may include or may be a liquid crystal display (LCD), and in some cases a fixed segment display or a dot matrix LCD display. If desired, the user interface 235 may be a touch screen LCD panel that functions as both display and keypad. In some instances, a touch screen LCD panel may be adapted to solicit values for a number of operating parameters and/or to receive such values, if desired. In some cases, the user interface 235 may optionally include the memory 232. In some cases, the user interface 235 may include one or more electromechanical input devices (e.g., a switch, a potentiometer, a rotary dial, a push button, etc.) for use in selecting a desired flow rate, vacuum level, and/or parameter.

In one example, the user interface 235 may include a graphical user interface communicatively coupled to the processor 215 and/or the memory 220, 232 via the data bus 217. The user interface 235 may be configured to allow a user to monitor and/or control one or more functions of the medical waste fluid collection cart 102 and/or the evacuation station 104. The user interface 235 may include one or more screens 237 that may be used to present information to a user. In some cases, a graphical user interface may be used to solicit entry of vacuum levels, flow rates and/or other information from a user via a touchscreen, a keypad, buttons on the medical waste fluid collection cart 102, and/or another electromechanical input device (e.g., a dial interface).

The communication interface 240 may include one or more communication interfaces for allowing the controller 210 to communicate with one or more other devices, such as via a communication link 247. For example, the communication interface 240 may include a communication interface that allows the medical waste fluid collection cart 102 to communicate with the evacuation station 104 via the coupler 146. In some cases, one or more connectors associated with the communication link 247 may be included within the coupler 146. In other cases, the communication link 247 may be separate from the coupler 146. In some cases, the communication interface 240 may include one or more wired and/or wireless communication interfaces, such as an Ethernet port, a wireless port, an RS-232 port, an RS-422 port, an RS-485 port, and the like. In such cases, the communication interface 240 may allow data entry, reprogramming, upgrading, debugging, and/or other operations to be done remotely, such as by an authorized user, via the communication link 247.

In some instances, the one or more sensors 265 in communication with the controller 210 may include a light sensor. In one example, the light sensor may be connected to the controller 210 and the controller 210 (e.g., processor 215, memory 220, and/or other components of the controller 210) may be configured to determine concentrations of fluid in a flow passing through one or more of the lid 124 or manifold, the suction tube 126, the container 112 and/or one or more other components of or connected to the fluid collection cart 102.

In some instances, the light sensor may be configured to detect one or more features related to light interacting with a fluid collected from a surgical site. Illustratively, the light sensor may be configured to detect one or more of opacity, color change, infrared (IR) absorption/transmission, IR reflection or other light related parameter of a fluid in a portion of the fluid collection system 100. In one example of a light sensor, the light sensor may include one or more light sources and one or more light detectors. Alternatively, or in addition, the light sensor may include a different configuration.

Surgical sites may include, among other fluids and loose particles (e.g., bone chips, tissue, etc.), waste fluid including blood and an irrigation fluid (e.g., saline or other irrigation fluid). When performing an operation that may include removing material from a surgical site, it may be desirable to know or estimate an amount of blood and/or irrigation fluid that has been removed from the surgical site. Such amounts may be determined by knowing a volume of the waste fluid that has been removed from the surgical site and knowing or estimating a concentration ratio of blood to irrigation fluid of the waste fluid making up the volume of the waste fluid that has been removed from the surgical site.

Waste fluid removed from a surgical site may be primarily made up of two materials (e.g., blood and an irrigation fluid) and a concentration of the waste fluid may range from 100% of a first fluid (e.g., blood or irrigation fluid) to 100% of a second fluid (e.g., the other of blood or irrigation fluid). Although particulates (e.g., bone chips, bone cement, tissue, and/or other particulates) may be present in the waste fluid, these particulates may be considered negligible for determining a concentration of the waste fluid.

A measurement of how a fluid allows light to pass through the fluid and/or reflects light may be used to determine a concentration ratio of materials making up the waste fluid. From knowing the materials of the waste fluid removed from the surgical site and general light absorbing and/or light reflecting properties of those materials, it may be possible to determine or estimate a concentration ratio of blood to irrigation fluid in the waste fluid. In one example, blood and an irrigation fluid (e.g., saline or other irrigation fluid) may have relatively distinct light absorbing and/or light reflecting properties over similar portions of a light spectrum, and thus, the concentration ratio of blood to irrigation fluid may be determined or estimated from measurements of light reflected by or passing through the waste fluid from a surgical site.

In some instances, a concentration ratio of the waste fluid removed from the surgical site may be an instantaneous determination (e.g., determined from a single measurement of light properties of the waste fluid). In one example, an instantaneous concentration ratio of waste fluid removed from the surgical site may be used when analyzing light properties of the waste fluid in a container 112 or other fluid holding portion of the cart 102. Alternatively, or in addition, to making an instantaneous determination of a concentration ratio, the concentration ratio of the waste fluid removed from the surgical site may be an average taken over a time period (e.g., determined from a plurality of measurements of light properties of waste fluid over a prescribed time period). In one example, an average concentration ratio may be utilized when analyzing light properties of the waste fluid as the waste fluid passes through the suction tubing 126 or passes through another portion of the cart 102.

Once a concentration ratio of blood versus irrigation fluid has been determined, an approximate amount of total blood removed from the surgical site may be calculated. In one example, a volume of the waste fluid removed from the surgical site may be determined from a sensor configured to measure a parameter that can be related to waste fluid volume of material removed (e.g., a sensor measuring flow rate of fluid, a sensor measuring a volume in a container 112, and/or a different sensor) and the determined volume may be multiplied by the identified concentration ratio of the fluid removed from surgical site to determine or estimate the total blood removed (e.g., blood loss) from the surgical site for a given procedure.

As referred to above, the light sensor may be configured to measure or detect one or more light related properties of a waste fluid collected from a surgical site. In one example, the light sensor may be configured to detect opacity of waste fluid collected from a surgical site by detecting changes in an intensity of light transmitted through the collected waste fluid at a location of the fluid collection system 100. This technique may utilize one or more light sources and one or more light detectors mounted substantially opposite one another or at another angle with respect to one another to measure an intensity of light from the light source that has passed through the waste fluid in the fluid collection system 100. Once the light sensor detects the intensity of the light that has passed through the waste fluid, the intensity may be provided to the controller 210 or other controller to determine a concentration ratio of the waste fluid collected from the surgical site based, at least in part, on the sensed light intensity.

Alternatively, or in addition, to a light sensor being configured to detect opacity of a collected waste fluid, a light sensor may be configured to detect a color change in a light from a light source by detecting color changes in a transmission of light from a light source through the collected waste fluid at a location of the fluid collection system 100. This technique may utilize one or more light sources and one or more light detectors mounted substantially opposite one another or at another angle with respect to one another to measure light passing through or reflected by the collected waste fluid. Illustratively, the light detector of the light sensor may detect a wavelength or other parameter of light from the light source that passes through or is reflected by the collected waste fluid to determine a color change of the light from the light source. Once the light sensor detects the color (e.g., wavelength) of the light that has passed through or that has been reflected by the waste fluid, the color may be provided to the controller to determine a concentration ratio of the waste fluid collected from the surgical site based, at least in part, on the sensed light color.

Alternatively, or in addition, to a light sensor being configured to detect opacity of a collected fluid or color of a light passing through or reflected by the collected waste fluid, a light sensor may be configured to detect infrared (IR) absorption/transmission of the collected waste fluid at a location of the fluid collection system 100. This technique may utilize one or more light sources (e.g., an infrared light source) of a known wavelength and one or more light detectors mounted substantially opposite one another or at another angle with respect to one another to measure a frequency of the light passing through the collected waste fluid. In some cases, two or more sources of light (e.g., diodes, lasers, or other sources of light) emitting different wavelengths of IR light may be utilized as the light source. When the collected waste fluid is primarily blood and irrigation fluid, a concentration ratio of blood to irrigation fluid may be determined as blood and irrigation fluid transmit and/or absorb distinctly different frequencies of IR light. Once the light sensor detects the IR absorption/transmission of the collected waste fluid (e.g., the intensity of the IR light passing through the collected fluid), the intensity may be provided to the controller to determine a concentration ratio of the waste fluid collected from the surgical site based, at least in part, on the sensed IR light intensity.

Alternatively, or in addition, to a light sensor being configured to detect opacity of a collected fluid, a color of a light passing through or reflected by the collected waste fluid, or IR absorption/transmission of properties of a collected waste fluid, a light sensor may be configured to detect IR reflection of the collected waste fluid at a location of the fluid collection system 100. This technique may utilize one or more light sources (e.g., an infrared light source) of a known wavelength and one or more light detectors mounted adjacent to the light source to measure a frequency of light from the light source that is reflected by the collected waste fluid. In some cases, two or more sources of light (e.g., diodes, lasers, or other sources of light) emitting different wavelengths of IR light may be utilized as the light source. When the collected waste fluid is primarily blood and irrigation fluid, a concentration ratio of blood to irrigation fluid may be determined as blood and irrigation fluid reflect distinctly different frequencies of IR light. Once the light sensor detects the intensity of the light reflected by the collected waste fluid, the intensity may be provided to the controller to determine a concentration ratio of the waste fluid collected from the surgical site based, at least in part, on the sensed IR light intensity.

Alternatively, or in addition, other techniques may be utilized to detect light or color properties waste fluid removed from a surgical site. In one example, an image or multiple images (e.g., video) of waste fluid removed from a surgical site may be analyzed (e.g., through pixel analysis or other analyses) to determine a concentration ratio of that fluid.

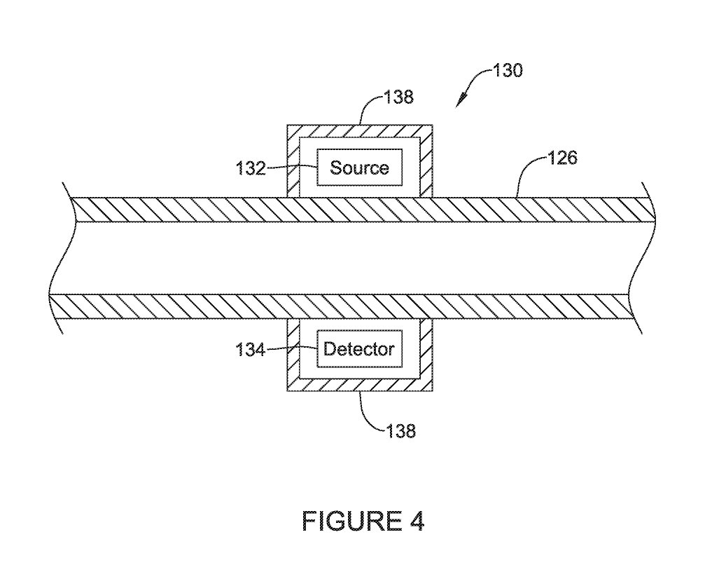

A light sensor configured to detect a measure or parameter related to a concentration ratio of waste fluid (e.g., blood and irrigation fluid, or other fluids) collected or removed from a surgical site may be located at any position on the fluid collection system 100. FIGS. 4-10 depict illustrative placements of a light sensor 130 on the fluid collection system 100. Illustrative placements of the light sensor 130 on the fluid collection system 100 may include, among other placements, placement on the suction tubing 126, placement on the fluid collection cart (e.g., adjacent one or more containers 112, or other location), placement on one or more of the containers 112, placement at or adjacent one or more lids 124 or manifolds, placement at or adjacent one or more auxiliary chambers 128, and/or placement at one or more other locations of the fluid collection system 100.

The suction tubing 126 may be configured from one or more materials. In one example, the suction tubing 126 may be configured from a material that is substantially transparent to light (e.g., visible light, infrared light, or light having a wavelength other than a wavelength of visible light or infrared light) from a light source of the light sensor, such that the suction tubing 126 has a limited, if any, effect on the light from the light source prior to the light reaching a light detector 134 of the light sensor. If the material of the suction tubing 126 does have an effect on the light from the light source, such effect can be known and taken into consideration when obtaining measurements from the light sensor 130.

A light source 132 of the light sensor 130 may be any source of light capable of emitting light to a fluid collected from a surgical site. Illustratively, the light source 132 may be capable of emitting light in one or more wavelengths. In one example, the light source 132 may include one or more light emitting diodes (LEDs), light emitting electrochemical cells (LECs), electroluminescent sheets, electroluminescent wires, lasers, and/or one or more other light sources capable of emitting light in visible wavelengths and/or non-visible wavelengths.

The light sensor 130 may be configured such that light from the light source 132 may emit from the light source 130 or be applied to fluid collected from the surgical site in a manner that provides constant light over a time period or pulsed light over a time period. Light from a light source 132 that is pulsed may be provided at any frequency and/or may be emitted or applied in a saw-toothed, square, or other wave pattern, which may facilitate detection of the light after the light has interacted with the collected fluid.

The light detector 134 of the light sensor 130 may be any detector configured to detect a light after the light has interacted with a waste fluid collected from a surgical site. Illustratively, the light detector 134 may be capable of detecting light in one or more wavelengths. For example, the light detector 134 may include active-pixel sensors, charge-coupled devices (CCD), LEDs, optical detectors, photoresistors (e.g., light dependent resistors (LDR), photovoltaic cells, photodiodes, photomultipliers, phototubes, phototransistors, and/or one or more other light detectors capable of detecting light in visible wavelengths and/or non-visible wavelengths.

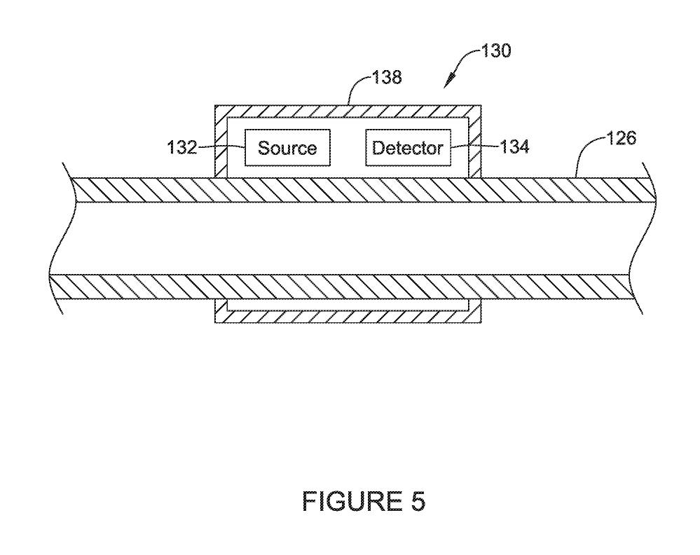

FIGS. 4 and 5 depict a light sensor 130 placed on the suction tube 126. The light sensor 130 may be placed at any location along the suction tube 126 including, but not limited to, adjacent an end of the suction tube 126 connected to the fluid collection cart 102, a position between a first end and an opposite second end of the suction tube 126, adjacent an end of the suction tube 126 that is configured to be positioned at a surgical site, within or adjacent a housing along the suction tube where the housing may include other sensors, other electronics, and/or one or more control mechanisms (e.g., a controller for turning on/off sensors, a controller for operating suction through the suction tube 126, and/or one or more other controllers), and/or at one or more other locations along the suction tube 126.

The light sensor 130 may include a housing 138 that may be configured to house one or more of a light source 132 and a light detector 134, while connecting to the suction tubing 126 in one or more manners. Illustratively, the light sensor 130 (e.g., the housing 138 or other portion of the light sensor 130) may be configured to connect to the suction tubing 126 through a friction fit, a snap fit, or other permanent, adjustable, or releasable connection type.

FIG. 4 depicts a light sensor 130 that may be configured to detect a characteristic of light from a light source 132 that may pass through a waste fluid collected from a surgical site. In one example, a light source 132 may be positioned along the suction tubing 126 (e.g., at or on the suction tubing 126 or at least partially spaced from the suction tubing 126) and a light detector 134 may be positioned along the suction tubing 126 (e.g., at or on the suction tubing 126 or at least partially spaced from the suction tubing 126) such that the light detector 134 may be positioned substantially opposite the light source 132. Such a configuration may allow the light sensor 130 to at least detect a characteristic (e.g., light intensity indicative of opacity of a waste fluid, wavelength change, light frequency indicative of IR absorption/transmission, and/or other characteristic) of a light from a light source that has passed through waste fluid collected from a surgical site and passing through a lumen of the suction tubing 126.

FIG. 5 depicts a light sensor 130 that may be configured to detect a characteristic of light from a light source 132 that may be reflected by a waste fluid collected from a surgical site. In one example, a light source 132 may be positioned along the suction tubing 126 (e.g., at or on the suction tubing 126 or at least partially spaced from the suction tubing 126) and a light detector 134 may be positioned along the suction tubing 126 (e.g., at or on the suction tubing 126 or at least partially spaced from the suction tubing 126) such that the light detector 134 may be positioned adjacent the light source 132. In instances when a light sensor 130 may be configured to detect light reflected by a waste fluid collected from a surgical site, the light detector 134 may be positioned to a distal or proximal side of the light source 132, to a radial side of the light source 132, around the light source 132, in an alternating arrangement with light source 132, and/or in one or more other arrangements relative to the light source 132. Such a configuration may allow the light sensor 130 to at least detect a characteristic (e.g., wavelength change, light frequency indicative of IR reflection, and/or other characteristic) of a light from a light source that has been reflected by waste fluid collected from a surgical site and passing through a lumen of the suction tubing 126.

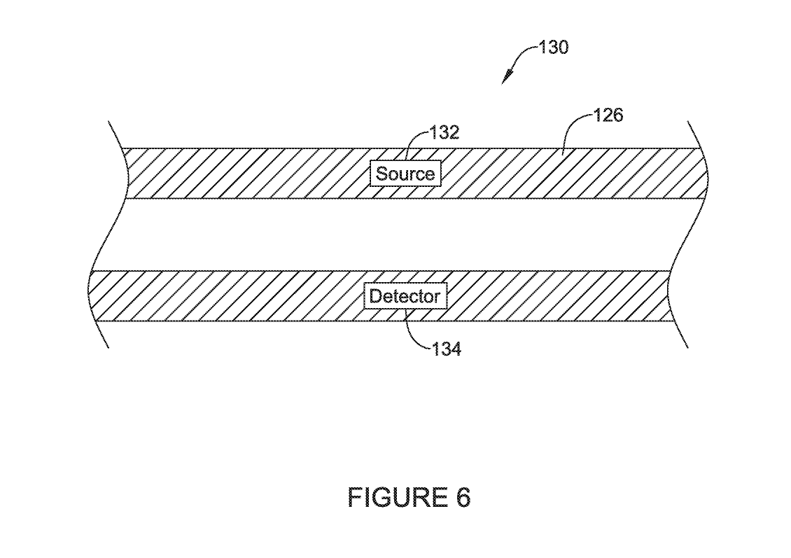

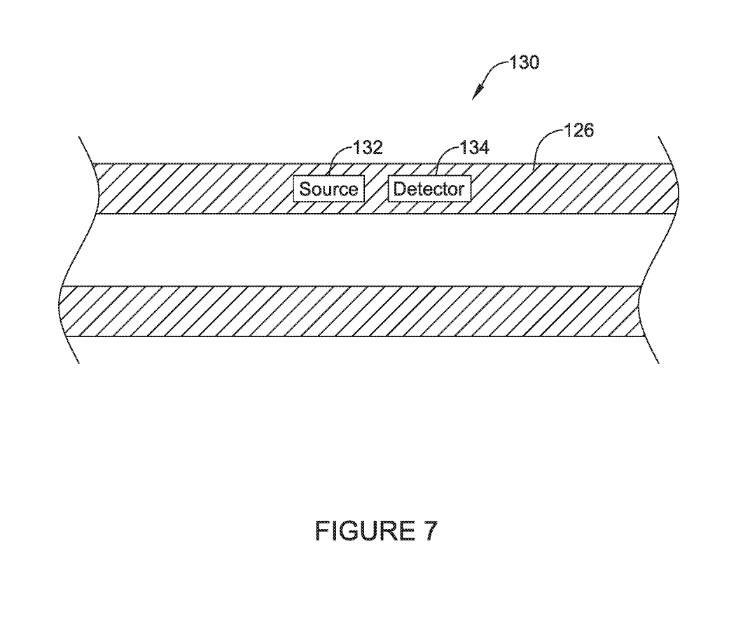

FIGS. 6 and 7 depict a light sensor 130 integrated in the suction tube 126. The light sensor 130 in the suction tube 126 may be placed at any location along the suction tube 126 including, but not limited to, adjacent an end of the suction tube 126 connected to the fluid collection cart 102, a position between a first end and an opposite second end of the suction tube 126, adjacent an end of the suction tube 126 that is configured to be positioned at a surgical site, within or adjacent to a connector or coupling of the suction tube 126, within or adjacent a housing along the suction tube where the housing may include other sensors, other electronics, and/or one or more control mechanisms (e.g., a controller for turning on/off sensors, a controller for operating suction through the suction tube 126, and/or one or more other controllers), and/or at one or more other locations along the suction tube 126.

The light sensor 130 located in the suction tube 126 may include a housing (not shown) that may be configured to house one or more of a light source 132 and a light detector 134 in the suction tube 126. Illustratively, the light sensor 130 (e.g., the housing or other portion of the light sensor 130) may be configured to form one or more walls of the suction tube 126 (e.g., an inner wall along the lumen of the suction tube 126 or an outer wall of the suction tube 126) and/or may be configured to be spaced from one or more walls of the suction tube 126.

A light sensor 130 located in the suction tube 126 may be powered and/or controlled by electronics in the sensor and/or by connecting the light sensor 130 to an external power source (e.g., through a port of the suction tube 126 in communication with the light sensor 130). In one example of a light sensor 130 in a suction tube 130, the suction tube 126 may include a port that mates with a port on the fluid collection cart 102 such that once the suction tube 126 is connected to the fluid connection cart 102 the light sensor 130 may be powered and/or connected to the controller 210. In another example of a light sensor 130 in a suction tube 126, a wired connection external the suction tube 126 may be connected to the light sensor 130 to power the light sensor 130 and/or connect the light sensor 130 to the controller 210. Alternatively, or in addition, the light sensor 130 may connect to a power source and/or the controller 210 in one or more other wired manners and/or may be wirelessly connected to a power source and/or controller 210.

When the suction tube 126 is a disposable suction tube, a light sensor 130 in the suction tube 126 may also be disposable. In one example, a light sensor 130 that is disposable may be made of materials that are inexpensive and that may be disposed of (e.g., recycled, discarded, etc.) with the suction tube 126 or in a manner similar to how the suction tube 126 may be disposed.

FIG. 6 depicts a light sensor 130 that may be configured to detect a characteristic of light from a light source 132 that may pass through a waste fluid collected from a surgical site. In one example, a light source 132 may be positioned in the suction tubing 126 and a light detector 134 may be positioned in the suction tubing 126 such that the light detector 134 may be positioned substantially opposite the light source 132. Such a configuration may allow the light sensor 130 to at least detect a characteristic (e.g., light intensity indicative of opacity of a waste fluid, wavelength change, light frequency indicative of IR absorption/transmission, and/or other characteristic) of a light from a light source that has passed through waste fluid collected from a surgical site and passing through a lumen of the suction tubing 126.

FIG. 7 depicts a light sensor 130 that may be configured to detect a characteristic of light from a light source 132 that may be reflected by a waste fluid collected from a surgical site. In one example, a light source 132 may be positioned in the suction tubing 126 and a light detector 134 may be positioned in the suction tubing 126 such that the light detector 134 may be positioned adjacent the light source 132. In instances when a light sensor 130 may be configured to detect light reflected by a waste fluid collected from a surgical site, the light detector 134 may be positioned to a distal or proximal side of the light source 132, to a radial side of the light source 132, around the light source 132, in an alternating arrangement with light source 132, and/or in one or more other arrangements relative to the light source 132. Such a configuration may allow the light sensor 130 to at least detect a characteristic (e.g., wavelength change, light frequency indicative of IR reflection, and/or other characteristic) of a light from a light source that has been reflected by waste fluid collected from a surgical site and passing through a lumen of the suction tubing 126.

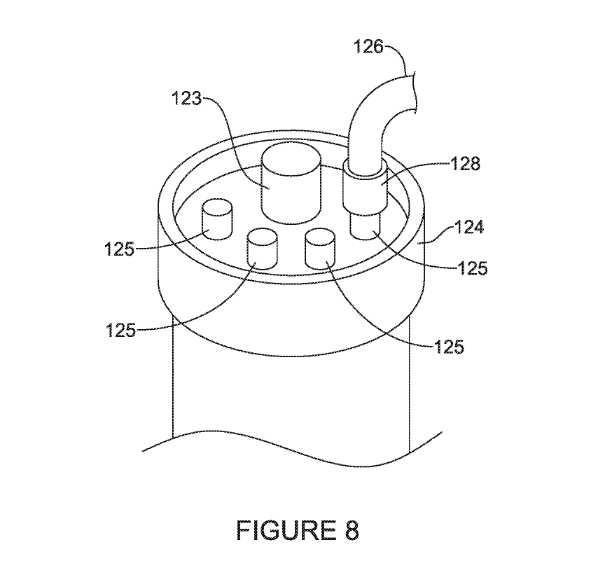

FIGS. 8-10 depict an auxiliary chamber 128 configured to house light sensor 130. Although the auxiliary chamber 128 is shown in FIGS. 6-8 as connecting to a suction port 125 and suction tubing 126, the auxiliary chamber 128 may connect to only the suction port 125, only the suction tubing 126, and/or one or more other parts of the fluid collection system 100.

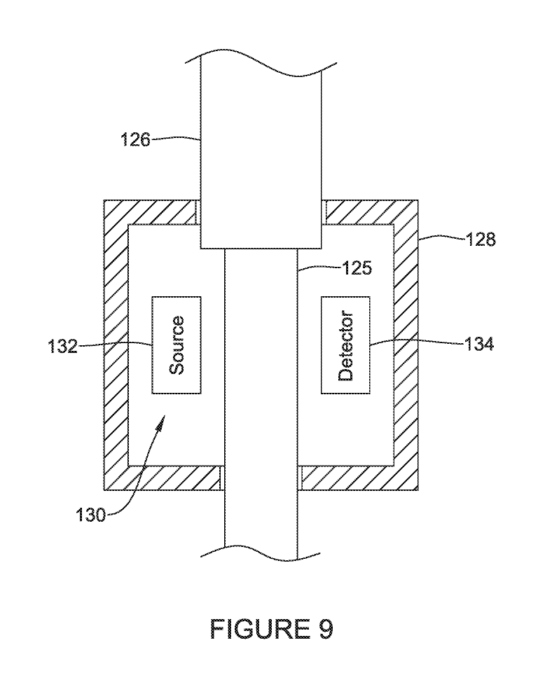

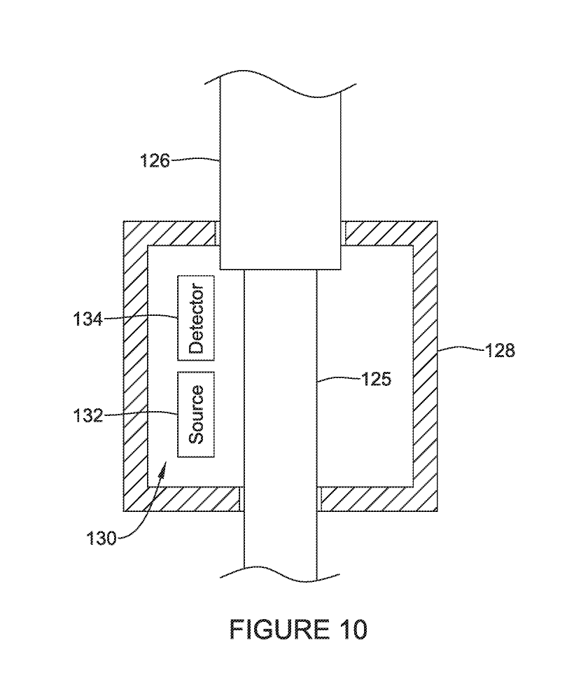

As shown in FIGS. 9 and 10, the auxiliary chamber 128 may be configured to receive the suction port 125 and the suction tube 126. In one example, the auxiliary chamber 128 may be configured to receive the suction port 125 through a different side of the auxiliary chamber 128 than a side through which the auxiliary chamber 128 receives the suction tubing 126 (e.g., suction port 125 may be received through an opening in the first end of the auxiliary chamber 128 and an opposite second end of the auxiliary chamber 128).

The auxiliary chamber 128 may connect to the suction port 125, the suction tubing 126, and/or one or more other portions of the fluid collection system 100 through one or more connection techniques. For example, the auxiliary chamber 128 may connect to one or more of the suction port 125 and the suction tubing 126 through a friction fit connection, a screw connection, a bayonet connection, a ball-detent connection, any other connection type, and/or any combination of connection types.

As shown in FIGS. 9 and 10, the light sensor 130 of the auxiliary chamber 128 may be configured to detect characteristics of light interacting with waste fluid collected from a surgical site as the waste fluid passes through the suction port 125 within the auxiliary chamber 128. Additionally, or alternatively, the light sensor 130 of the auxiliary chamber 128 may be configured to detect characteristics of light interacting with fluid collected from the surgical site as the waste fluid passes through the suction tubing 126 or as the waste fluid passes through or is contained in other portions of the fluid collection system 100.

FIG. 9 depicts a light sensor 130 that may be configured to detect a characteristic of light from a light source 132 that may pass through a waste fluid collected from a surgical site. In one example, a light source 132 may be positioned along the suction port 125 (e.g., at or on the suction tubing 126 or at least partially spaced from the suction tubing 126) and a light detector 134 may be positioned along the suction port 125 (e.g., at or on the suction tubing 126 or at least partially spaced from the suction tubing 126) such that the light detector 134 may be positioned substantially opposite the light source 132. Such a configuration may allow the light sensor 130 to at least detect a characteristic (e.g., light intensity indicative of opacity of a waste fluid, wavelength change, light frequency indicative of IR absorption/transmission, and/or other characteristic) of a light from the light source 132 that has passed through waste fluid collected from a surgical site.

FIG. 10 depicts a light sensor 130 that may be configured to detect a characteristic of light from a light source 132 that may be reflected by a waste fluid collected from a surgical site. In one example, a light source 132 may be positioned along the suction port 125 (e.g., at or on the suction tubing 126 or at least partially spaced from the suction tubing 126) and a light detector 134 may be positioned along the suction port 125 (e.g., at or on the suction tubing 126 or at least partially spaced from the suction tubing 126) such that the light detector 134 may be positioned adjacent the light source 132. In instances when a light sensor 130 may be configured to detect light reflected by a waste fluid collected from a surgical site, the light detector 134 may be positioned to a distal or proximal side of the light source 132, to a radial side of the light source 132, around the light source 132, in an alternating arrangement with light source 132, and/or in one or more other arrangements relative to the light source 132. Such a configuration may allow the light sensor 130 to at least detect a characteristic (e.g., wavelength change, light frequency indicative of IR reflection, and/or other characteristic) of a light from a light source that has been reflected by waste fluid collected from a surgical site.

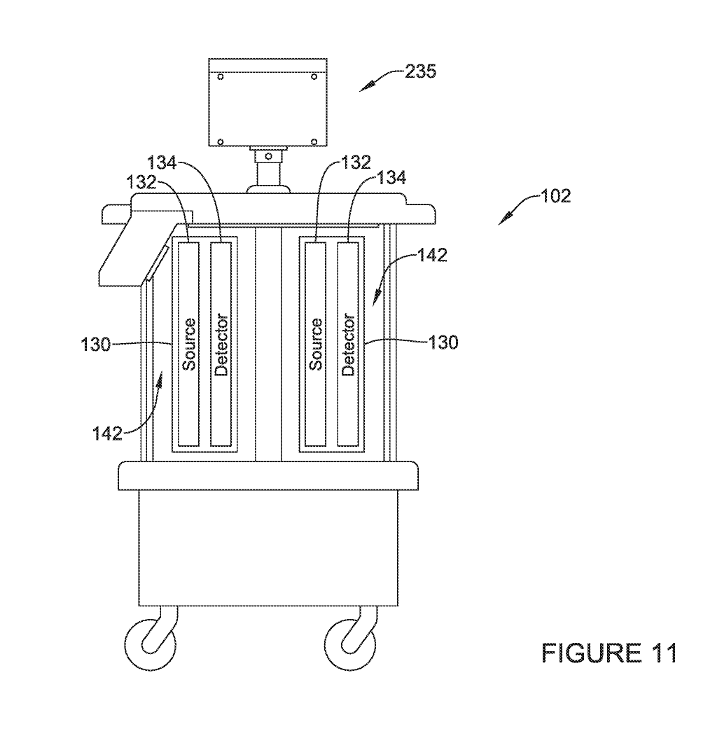

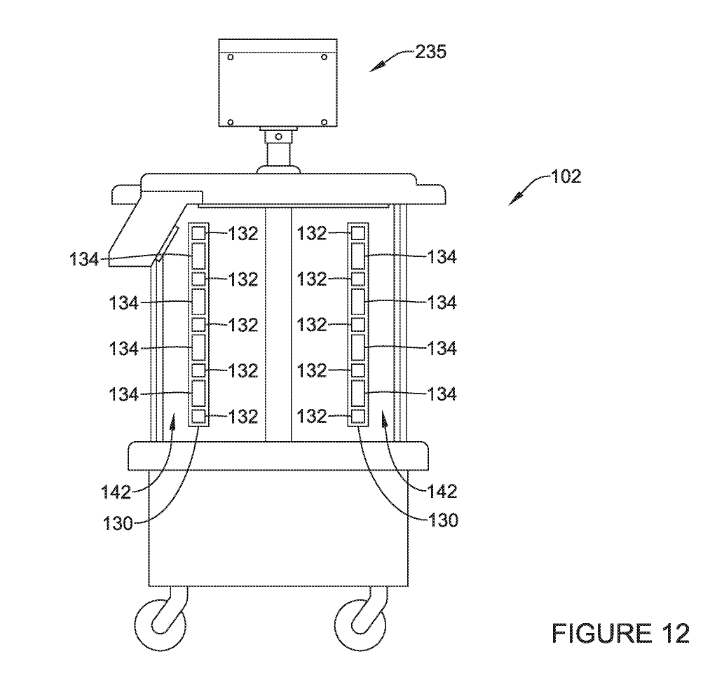

FIGS. 11 and 12 depict a fluid collection cart 102 that includes a user interface 235 and one or more designated spaces 142 for receiving a container 112 (e.g., two designated spaces 142 in FIGS. 11 and 12). A light sensor 130 may be positioned within the spaces 142 for receiving a container 112. Such a light sensor 130 may be in or on or otherwise connected to a wall of the fluid collection cart 102, such that light may be emitted from a light source 132 of the light sensor 130 to collected waste fluid held within a container 112 in the space 142 and light reflected by the collected waste fluid held within the container 112 in the space 142 may be detected by the light detector 134. In instances when a light sensor 130 may be configured to detect light reflected by a waste fluid collected from a surgical site, the light detector 134 may be positioned to a distal or proximal side of the light source 132, to a radial side of the light source 132 (e.g., as in FIG. 11), around the light source 132, in an alternating arrangement with light source 132 (e.g., as in FIG. 12), and/or in one or more other arrangements relative to the light source 132. Such a configuration may allow the light sensor 130 to at least detect a characteristic (e.g., wavelength change, light frequency indicative of IR reflection, and/or other characteristic) of a light from a light source that has been reflected by waste fluid collected from a surgical site.

Alternatively, or in addition to, the light sensor 130 adjacent a space 142 configured to receive a container 112 being configured to detect light reflected by a collected waste fluid, the light sensor 130 may be configured on the fluid collection cart 102 to detect a characteristic of light from a light source 132 that may pass through waste fluid collected from a surgical site and located in the container 112. In one example, a light source 132 may be positioned along a wall of the fluid collection cart 102 at least partially defining the space 142 for receiving the container 112 and a light detector 134 may be positioned along a wall of the fluid collection cart 102 (e.g., the same wall as or a different wall than the wall on which the light source 132 may be located) such that the light detector 134 may be positioned substantially opposite the light source 132. Such a configuration may allow the light sensor 130 to at least detect a characteristic (e.g., light intensity indicative of opacity of a waste fluid, wavelength change, light frequency indicative of IR absorption/transmission, and/or other characteristic) of a light from the light source 132 that has passed through waste fluid collected from a surgical site.

In some instances, one or more light sensors 130 may be located on the container 112 to detect light passing through and/or reflected by waste fluid collected from a surgical site and located in the container 112. Such a light sensor 130 may include a light source 132 and a light detector 134 configured with respect to one another in manners similar or dissimilar to the light sensors 130 described with respect to FIGS. 4-12. The light sensors 130 on the container 112 may include a wired or wireless connection to the controller 210 or other controller.

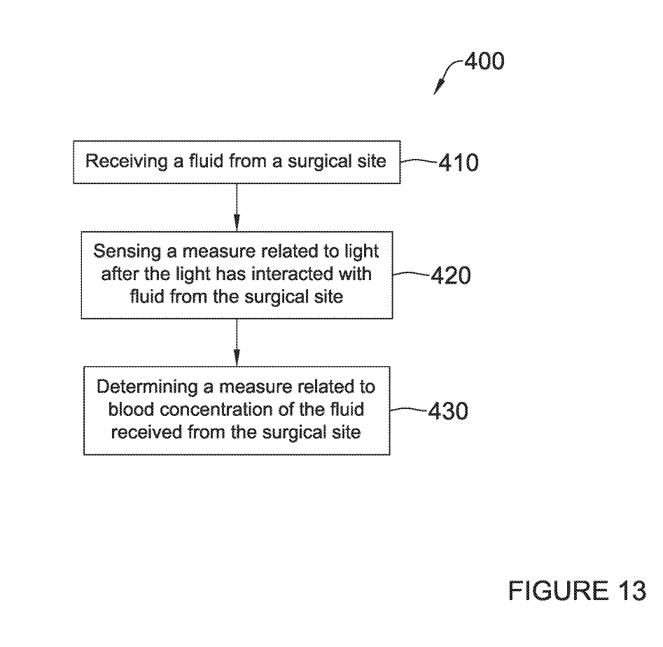

In operation (400, as shown in FIG. 13), once fluid collection system 100 receives (410) waste fluid from a surgical site, a light sensor 130 measures or senses (420) a measure of a parameter related to a characteristic of a light that has interacted with waste fluid collected from a surgical site as the waste fluid is received or while the waste fluid is being contained in container 112. The light sensor 130 may communicate the measurement to the controller 210 or other controller (e.g., a controller at or in the light sensor 130) for processing. The memory 220 of the controller 210 or other memory may include measurement correlations, algorithms, look-up tables, and/or other data that relates the measured parameter to a concentration of the waste fluid collected from the surgical site. The controller 210 may then calculate or determine (430) a measure (e.g., an estimated average or a precise average having a predetermined accuracy) related to the concentration ratio (e.g., a concentration ratio of blood to irrigation fluid (e.g., saline or other fluid)) of the collected waste fluid over a period of time or calculate or determine the concentration of the collected waste fluid at a particular time from the measured parameter and the stored measurement correlations, algorithms, look-up tables, and/or other data. The calculated or determined concentrations of the collected waste fluid may be stored or communicated to a memory and/or display.

Alternatively, or in addition, to storing or communicating the calculated or determined concentration of the collected waste fluid, the calculated or determined concentrations may be further processed. In one example, the controller 210 or other controller may obtain volume and/or flow rate related information of the waste fluid collected from the surgical site and using the calculated or determined concentration of the collected waste fluid, calculate a volume of a particular fluid in the waste fluid collected from the surgical site. Illustratively, the controller 210 (e.g., processor 215) may be configured to determine an amount of blood in the waste fluid collected from the surgical site (e.g., a measurement of blood loss from the surgical site) based on an average blood concentration of the collected waste fluid over time (e.g., during a time from a start of a surgical procedure and while the collected waste fluid travels to a container 112) or an instantaneous blood concentration calculation of waste fluid collected in a storage container 112.

For example, the controller 210 may collect data related to the concentration ratio (e.g., the concentration ratio of blood to irrigation fluid) of a quantity of the collected waste fluid flowing past the light sensor 130 at a plurality of times (e.g., time N, time N+1, time N+2, time N+3, etc.), and using the collected data, the controller 210 may determine the estimated total amount (e.g., volume and/or concentration) of blood and/or irrigation fluid collected over the time period (e.g., between time N to time N+1, or between time N to time N+2, or between time N to time N+3, etc.) in which the waste fluid was collected. For example, the controller 210 may determine an average concentration of blood from the plurality of concentration measurements taken at time N, time N+1, time N+2, time N+3, etc. and/or the controller 210 may determine a total estimated volume of blood collected by summing a calculated volume of blood flowing past the light sensor and collected during each time interval. For instance, by measuring the concentration ratio of a quantity of waste fluid flowing past the light sensor during a time interval and measuring the volume of waste fluid flowing past the light sensor during the same time interval (e.g., by measuring the flow rate of the waste fluid past the light sensor), the controller 210 can determine the quantity (volume) of blood and/or irrigation fluid in the collected volume of waste fluid during that time interval. The calculated data from multiple time intervals may be compiled (e.g., the volumes of blood may be summed and/or concentrations of blood may be averaged) to determine the volume and/or concentration of blood (or irrigation fluid) collected during the entire time period of collecting waste fluid. The controller 210 may be able to determine instances in which no waste fluid is flowing past the light sensor during the period of collecting waste fluid, and thus adjust the calculated quantity of collected blood and/or concentration of blood accordingly. The calculated or determined concentrations of the collected waste fluid (including the estimated total amount (e.g., volume and/or concentration) of blood and/or irrigation fluid collected) may be stored or communicated to a memory and/or display.

In one example operation of determining blood loss from a surgical site, the light sensor 130 and controller 210 may determine and, optionally, display an irrigation fluid to blood ratio of the waste fluid, a total volume of collected waste fluid, a total volume of collected blood or other quantity of blood calculation, a total volume of collected irrigation fluid or other quantity of irrigation fluid calculation, and/or one or more other calculations related to the collected waste fluid. Software in the controller 210 may continuously sample the irrigation fluid to blood ratio of the collected waste fluid and translate the irrigation fluid to blood ratio and/or other calculations related to collected waste fluid to a reportable value.

Example reportable values of the irrigation fluid to blood ratio and/or other calculations related to collected waste fluid may include, but are not limited to, values along a scale from 1-10, positioning a moving bar along a continuum, a color of a color scale, a value of an estimated or exact irrigation fluid to blood ratio, a value of an estimated or exact volume of blood collected, a value of an estimate or exact percentage of blood collected, a value of an estimated or exact volume of irrigation fluid collected, a value of an estimated or exact percentage of irrigation fluid collected, and/or other reportable value. Optionally, the controller 210 may display the reportable value on a display, report the reportable value to one or more other controllers or computing devices, initiate an alarm (e.g., an audio, visual, and/or other sensory alarm) after the reportable value reaches a particular threshold, lock-out vacuum pressure after the reportable value reaches a particular threshold, and/or take one or more other actions.

In some instances, the reportable value may be displayed on a user interface from which a user may take an action in response to observing the reportable value. In one example, a user interface may display the reportable value and include selectable buttons or options (e.g., via a touch screen or buttons adjacent a display) that may be selected to cause the fluid collection system 100 to take an action (e.g., increase suction, decrease suction, etc.)

In an example of initiating an alarm after a reportable value reaches a particular threshold, the controller 210 may initiate and/or sound an alarm (e.g., an audio alarm, a visual alarm, other sensory alarms, a control signal, and/or other alarm) when a measure related to blood loss from a surgical site (e.g., a blood concentration in, total blood volume of, and/or other indication of blood level in collected fluid) passes an alarm threshold level (e.g., is above or below the threshold level). In some instances, such an alarm may indicate that the fluid collection system 100 is collecting more blood from a surgical site than is expected, indicate that the fluid collection system 100 may be mistakenly connected to an incorrect surgical tube (e.g., a chest tube or other surgical tube), and/or indicate there has been an inadvertent rupture within a patient in which the fluid collection system 100 is being used.

The alarm may be configured to alert users of the fluid collection system 100 of an unusually high amount (e.g., quantity and/or concentration) of blood in the collected fluid and/or suggest one or more actions to take in responding to the alarm being initiated. In addition, or alternatively, when an alarm is triggered, the controller 210 may be configured to automatically take an action. For example, when an alarm is triggered, the controller 210 may be configured to cease suction through the suction tube 126 due to a severity of the situation when the blood concentration and/or blood quantity alarm threshold is exceeded.

One or more alarm thresholds may be set by a user interacting with buttons and/or a display on a user interface 235 associated with the fluid collection cart 102. Alternatively, or in addition, alarm thresholds may be set through a user interface that is spaced from the fluid collection cart 102 over a wired and/or wireless connection, may be set by a manufacturer of the fluid collection cart 102, and/or set in one or more other manners.

In some cases, the alarm threshold may be set to a level such that if a blood quantity or blood concentration measurement of collected fluid is above that preset level, the blood concentration and/or quantity of blood in the collected fluid is above a normal or acceptable level that is to be expected for a particular surgical use or application of the fluid collection cart 102. As such, the threshold level may be dictated and/or adjusted depending on the use of the fluid collection cart 102. In such instances, the user interface 235 may provide an option for a user to enter or select a procedure from a plurality of pre-programed procedures (e.g., selected from a list of pre-programed procedures on the display of the user interface 235) in which the fluid collection cart 102 will be used and the alarm threshold may be automatically set to a level associated with the entered or selected procedure. Each pre-programed procedure may have an associated programed threshold level above which the alarm or warning would be triggered. Alternatively, or in addition, a threshold level may be adjusted from the automatically set level (e.g., pre-programed level) and/or may otherwise be set by a user (e.g., manually selected or entered), as desired. However, in some cases, an alarm threshold lockout may be provided to prevent a user from inadvertently or otherwise changing preset or automatically set alarm threshold levels.

After a first surgical procedure has been concluded and the fluid collection cart 102 is to be used for a second surgical procedure, the collected waste fluid concentration and/or blood loss data may be cleared or stored for later reference and then zeroed with respect to a display such that collected fluid concentration, blood loss data (e.g., volume of collected blood), volume of irrigation fluid collected, and/or other data may be calculated or determined for a second surgical procedure without having to empty the container(s) 112 of the fluid collection cart 102 between procedures and/or uses. Such a configuration may allow the fluid collection cart to be used in multiple surgical procedures without a need to reset the software of the controller 210 and/or without having to empty the container(s) 112 of the fluid collection cart 102.

Although the sensor 130 and controller 210 of the fluid collection cart has been described for use in determining blood loss during a surgical procedure, the sensor 130 and controller 210 may be used in one or more other procedures. In one example, the sensor 130 and controller 210 may be utilized in cleaning procedures, flushing procedures, and/or one or more other medical or non-medical related procedures.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

### Various Notes & Examples

Example 1 can include or use subject matter such as a fluid collection system, comprising: a container; a suction line having a lumen extending through the suction line, the suction line is in communication with the container to deposit waste fluid removed from a surgical site and passing through the lumen of the suction line into the container; and a light sensor configured to sense a measure related to concentration of blood in the waste fluid removed from the surgical site.

Example 2 can include, or can optionally be combined with the subject matter of Example 1, to optionally include a light sensor that is adjacent the suction line and configured to sense a measure related to the concentration of the blood in the waste fluid removed from the surgical site as the waste fluid passes through the lumen of the suction line.

Example 3 can include, or can optionally be combined with the subject matter of one or any combination of Examples 1 or 2 to optionally include a light sensor that is configured to at least partially engage the suction line at a position between an end of the suction line adjacent the surgical site and an end of the suction line adjacent the container.

Example 4 can include, or can optionally be combined with the subject matter of one or any combination of Examples 1 through 3 to optionally include a light sensor that is integrated in the suction line.

Example 5 can include, or can optionally be combined with the subject matter of one or any combination of Examples 1 through 4 to optionally include a light sensor and the suction line are disposable.

Example 6 can include, or can optionally be combined with the subject matter of one or any combination of Examples 1 through 5 to optionally include a cart configured to at least partially support the container; and wherein the light sensor is at least partially positioned at the cart adjacent the container.

Example 7 can include, or can optionally be combined with the subject matter of one or any combination of Examples 1 through 6 to optionally include a light sensor that is configured to sense a measure related to the concentration of blood in a volume of waste fluid deposited in the container.

Example 8 can include, or can optionally be combined with the subject matter of one or any combination of Examples 1 through 7 to optionally include a light sensor that can comprise: a light source; and a light detector configured to detect light from the light source to sense a measure related to the concentration of blood in the waste fluid removed from the surgical site.

Example 9 can include, or can optionally be combined with the subject matter of one or any combination of Examples 1 through 8 to optionally include a light detector that is configured to detect an intensity of light from the light source that is transmitted through the waste fluid from the surgical site.

Example 10 can include, or can optionally be combined with the subject matter of one or any combination of Examples 1 through 9 to optionally include a light detector that is configured to detect a change in color of light from the light source that is transmitted through the waste fluid from the surgical site.

Example 11 can include, or can optionally be combined with the subject matter of one or any combination of Examples 1 through 10 to optionally include a light source that can be an infrared light source; and a light detector that can be configured to detect a frequency of light from the infrared light source that is transmitted through the waste fluid from the surgical site.

Example 12 can include, or can optionally be combined with the subject matter of one or any combination of Examples 1 through 11 to optionally include a light source that can be an infrared light source; and a light detector that can be configured to detect a frequency of light from the infrared light source that is reflected by the waste fluid from the surgical site.

Example 13 can include, or can optionally be combined with the subject matter of one or any combination of Examples 1 through 12 to optionally include memory configured to store correlations between the sensed measure related to blood concentration and blood concentration in waste fluid from the surgical site; a processor in communication with the memory and the light sensor; and wherein the processor is configured to estimate an average blood concentration of the removed waste fluid over a period of time based at least in part on sensed measures related to blood concentration and the stored correlations between the sensed measure related to blood concentration and blood concentration in waste fluid received from a surgical site.

Example 14 can include, or can optionally be combined with the subject matter of one or any combination of Examples 1 through 13 to optionally include a controller in communication with the light sensor to receive sensed measures related to concentration of blood in the waste fluid removed from the surgical site; and wherein the controller is configured to initiate an alarm if the received sensed measures related to concentration of blood in the waste fluid removed from a surgical site exceeds an alarm threshold.

Example 15 can include, or can optionally be combined with the subject matter of one or any combination of Examples 1 through 14 to optionally include a controller that is configured to stop suction in the suction line when the alarm is initiated.

Example 16 can include or use subject matter such as a fluid collection system, comprising: a fluid collection cart including at least one container; a suction line having a lumen, the suction line is configured to extend from a surgical site to one or more of the at least one container and the lumen is configured to receive waste fluid from the surgical site; and a sensor configured to sense a measure related to concentration of blood in the waste fluid received from the surgical site as the waste fluid is received.

Example 17 can include, or can optionally be combined with the subject matter of Example 16, to optionally include a suction line that is disposable and the sensor is integrated in the suction line to be disposed of with the disposable suction line.

Example 18 can include, or can optionally be combined with the subject matter of one or any combination of Examples 16 or 17 to optionally include memory configured to store correlations between the sensed measure related to blood concentration and blood concentration in waste fluid from a surgical site; a processor in communication with the memory and the light sensor; and wherein the processor is configured to estimate an average blood concentration of the received waste fluid over a period of time based at least in part on sensed measures related to blood concentration and the stored correlations between the sensed measure related to blood concentration and blood concentration in waste fluid from a surgical site.

Example 19 can include, or can optionally be combined with the subject matter of one or any combination of Examples 16 through 18 to optionally include a processor that can be configured to estimate a total blood loss from the surgical site over the period of time based at least partially on the estimated average blood concentration of the waste fluid received from the surgical site.

Example 20 can include, or can optionally be combined with the subject matter of one or any combination of Examples 16 through 19 to optionally include a sensor that can be configured to detect light that has passed through the waste fluid received from the surgical site.

Example 21 can include, or can optionally be combined with the subject matter of one or any combination of Examples 16 through 20 to optionally include a sensor that can be configured to detect light that has been reflected by the waste fluid received from the surgical site.

Example 22 can include or use subject matter such as a method of analyzing waste fluid collected from a patient during a surgical procedure on the patient, the method comprising: receiving waste fluid from a surgical site; sensing a measure related to light after the light has interacted with the waste fluid received from the surgical site as the waste fluid is received; and determining a measure related to blood concentration of the waste fluid received from the surgical site as the waste fluid is received, wherein the measure related to blood concentration is based at least in part on the measure related to light sensed after the light has interacted with the waste fluid received from the surgical site.

Example 23 can include, or can optionally be combined with the subject matter of Example 22, to optionally include determining a measure related to blood loss from a surgical site based at least in part on the determined measure related to blood concentration of the waste fluid received from the surgical site.

Example 24 can include, or can optionally be combined with the subject matter of one or any combination of Examples 22 or 23 to optionally include initiating an alarm if the measure related to blood from a surgical site exceeds a threshold level.

Example 25 can include, or can optionally be combined with the subject matter of one or any combination of Examples 22 through 24 to optionally include sensing a measure related to light interacting with the waste fluid received from the surgical site to include detecting light that has passed through the waste fluid received from the surgical site.

Example 26 can include, or can optionally be combined with the subject matter of one or any combination of Examples 22 through 25 to optionally include sensing a measure related to light interacting with the waste fluid received from the surgical site to include detecting light reflected by the waste fluid received from the surgical site.

Example 27 can include, or can optionally be combined with the subject matter of one or any combination of Examples 22 through 26 to optionally include sensing a measure related to light interacting with the waste fluid received from the surgical site to include: transmitting a light from a light source through the fluid received from the surgical site; and sensing a measure related to the intensity of light transmitted through the waste fluid received from the surgical site.

Example 28 can include, or can optionally be combined with the subject matter of one or any combination of Examples 22 through 27 to optionally include sensing a measure related to light interacting with the waste fluid received from the surgical site to include: transmitting a light from a light source through the waste fluid received from the surgical site; and sensing a measure related to the wavelength of light transmitted through the waste fluid received from the surgical site.

Example 29 can include, or can optionally be combined with the subject matter of one or any combination of Examples 22 through 28 to optionally include sensing a measure related to light interacting with the waste fluid received from the surgical site to include: transmitting a light from an infrared light source through the waste fluid received from the surgical site; and sensing a measure related to the frequency of light transmitted through the waste fluid received from the surgical site.

Example 30 can include, or can optionally be combined with the subject matter of one or any combination of Examples 22 through 29 to optionally include sensing a measure related to light interacting with the waste fluid received from the surgical site to include: transmitting a light from an infrared light source through the waste fluid received from the surgical site; and sensing a measure related to the frequency of light reflected by the waste fluid received from the surgical site.

Example 31 can include, or can optionally be combined with the subject matter of one or any combination of Examples 22 through 30 to optionally include sensing a measure related to light interacting with the waste fluid received from the surgical site to include: transmitting a light from a laser light source through the waste fluid received from the surgical site; and sensing a measure related to the intensity of light transmitted through the waste fluid received from the surgical site.

Example 32 can include, or can optionally be combined with the subject matter of one or any combination of Examples 22 through 31 to optionally include sensing a measure related to light interacting with the waste fluid received from the surgical site to include: transmitting a light from a laser light source through the waste fluid received from the surgical site; and sensing a measure related to the intensity of light reflected by the waste fluid received from the surgical site.

Example 33 can include, or can optionally be combined with the subject matter of one or any combination of Examples 22 through 32 to optionally include sensing a measure related to light interacting with the waste fluid received from the surgical site to include: transmitting a light from a laser light source through the waste fluid received from the surgical site; and sensing a measure related to the frequency of light transmitted through the waste fluid received from the surgical site.

Example 34 can include, or can optionally be combined with the subject matter of one or any combination of Examples 22 through 33 to optionally include sensing a measure related to light interacting with the waste fluid received from the surgical site to include: transmitting a light from a laser light source through the waste fluid received from the surgical site; and sensing a measure related to the frequency of light reflected by the waste fluid received from the surgical site.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A fluid collection system, comprising:
a container;
a suction line having a lumen extending through the suction line, the suction line is in communication with the container to deposit waste fluid removed from a surgical site and passing through the lumen of the suction line into the container; and
a light sensor configured to sense a measure related to concentration of blood in the waste fluid removed from the surgical site.

2. The system of claim 1, wherein the light sensor is adjacent the suction line and configured to sense a measure related to the concentration of the blood in the waste fluid removed from the surgical site as the waste fluid passes through the lumen of the suction line.

3. The system of claim 2, wherein the light sensor is configured to at least partially engage the suction line at a position between an end of the suction line adjacent the surgical site and an end of the suction line adjacent the container.

4. The system of claim 2, wherein the light sensor is integrated in the suction line.

5. The system of claim 4, wherein the light sensor and the suction line are disposable.

6. The system of claim 1, further comprising:
a cart configured to at least partially support the container; and
wherein the light sensor is at least partially positioned at the cart adjacent the container.

7. The system of claim 6, wherein the light sensor is configured to sense a measure related to the concentration of blood in a volume of waste fluid deposited in the container.

8. The system of claim 1, wherein the light sensor comprises:
a light source; and
a light detector configured to detect light from the light source to sense a measure related to the concentration of blood in the waste fluid removed from the surgical site.

9. The system of claim 8, wherein the light detector is configured to detect an intensity of light from the light source that is transmitted through the waste fluid from the surgical site.

10. The system of claim 8, wherein the light detector is configured to detect a change in color of light from the light source that is transmitted through the waste fluid from the surgical site.

11. The system of claim 8, wherein:
the light source is an infrared light source; and
the light detector is configured to detect a frequency of light from the infrared light source that is transmitted through the waste fluid from the surgical site.

12. The system of claim 8, wherein:
the light source is an infrared light source; and
the light detector is configured to detect a frequency of light from the infrared light source that is reflected by the waste fluid from the surgical site.

13. The system of claim 1, further comprising:
memory configured to store correlations between the sensed measure related to blood concentration and blood concentration in waste fluid from the surgical site;
a processor in communication with the memory and the light sensor; and
wherein the processor is configured to estimate an average blood concentration of the removed waste fluid over a period of time based at least in part on sensed measures related to blood concentration and the stored correlations between the sensed measure related to blood concentration and blood concentration in waste fluid received from a surgical site.

14. The system of claim 1, further comprising:
a controller in communication with the light sensor to receive sensed measures related to concentration of blood in the waste fluid removed from the surgical site; and
wherein the controller is configured to initiate an alarm if the received sensed measures related to concentration of blood in the waste fluid removed from a surgical site exceeds an alarm threshold.

15. The system of claim 14, wherein the controller is configured to stop suction in the suction line when the alarm is initiated.

16. A fluid collection system, comprising:
a fluid collection cart including at least one container;
a suction line having a lumen, the suction line is configured to extend from a surgical site to one or more of the at least one container and the lumen is configured to receive waste fluid from the surgical site; and
a sensor configured to sense a measure related to concentration of blood in the waste fluid received from the surgical site as the waste fluid is received.

17. The system of claim 16, wherein the suction line is disposable and the sensor is integrated in the suction line to be disposed of with the disposable suction line.

18. The system of claim 16, further comprising:
memory configured to store correlations between the sensed measure related to blood concentration and blood concentration in waste fluid from a surgical site;
a processor in communication with the memory and the light sensor; and
wherein the processor is configured to estimate an average blood concentration of the received waste fluid over a period of time based at least in part on sensed measures related to blood concentration and the stored correlations between the sensed measure related to blood concentration and blood concentration in waste fluid from a surgical site.

19. The system of claim 18, wherein the processor is configured to estimate a total blood loss from the surgical site over the period of time based at least partially on the estimated average blood concentration of the waste fluid received from the surgical site.

20. The system of claim 16, wherein the sensor is configured to detect light that has passed through the waste fluid received from the surgical site.

21. The system of claim 16, wherein the sensor is configured to detect light that has been reflected by the waste fluid received from the surgical site.

22. A method of analyzing waste fluid collected from a patient during a surgical procedure on the patient, the method comprising:
receiving waste fluid from a surgical site;
sensing a measure related to light after the light has interacted with the waste fluid received from the surgical site as the waste fluid is received; and
determining a measure related to blood concentration of the waste fluid received from the surgical site as the waste fluid is received, wherein the measure related to blood concentration is based at least in part on the measure related to light sensed after the light has interacted with the waste fluid received from the surgical site.

23. The method of claim 22, further comprising
determining a measure related to blood loss from a surgical site based at least in part on the determined measure related to blood concentration of the waste fluid received from the surgical site.

24. The method of claim 22, further comprising:
initiating an alarm if the measure related to blood from a surgical site exceeds a threshold level.

25. The method of claim 22, wherein sensing a measure related to light interacting with the waste fluid received from the surgical site includes detecting light that has passed through the waste fluid received from the surgical site.

26. The method of claim 22, wherein sensing a measure related to light interacting with the waste fluid received from the surgical site includes detecting light reflected by the waste fluid received from the surgical site.

27. The method of claim 22, wherein sensing a measure related to light interacting with the waste fluid received from the surgical site includes:
transmitting a light from a light source through the fluid received from the surgical site; and
sensing a measure related to the intensity of light transmitted through the waste fluid received from the surgical site.

28. The method of claim 22, wherein sensing a measure related to light interacting with the waste fluid received from the surgical site includes:
transmitting a light from a light source through the waste fluid received from the surgical site; and
sensing a measure related to the wavelength of light transmitted through the waste fluid received from the surgical site.

29. The method of claim 22, wherein sensing a measure related to light interacting with the waste fluid received from the surgical site includes:
transmitting a light from an infrared light source through the waste fluid received from the surgical site; and
sensing a measure related to the frequency of light transmitted through the waste fluid received from the surgical site.

30. The method of claim 22, wherein sensing a measure related to light interacting with the waste fluid received from the surgical site includes:
transmitting a light from an infrared light source through the waste fluid received from the surgical site; and
sensing a measure related to the frequency of light reflected by the waste fluid received from the surgical site.

31. The method of claim 22, wherein sensing a measure related to light interacting with the waste fluid received from the surgical site includes:
transmitting a light from a laser light source through the waste fluid received from the surgical site; and
sensing a measure related to the intensity of light transmitted through the waste fluid received from the surgical site.

32. The method of claim 22, wherein sensing a measure related to light interacting with the waste fluid received from the surgical site includes:
transmitting a light from a laser light source through the waste fluid received from the surgical site; and
sensing a measure related to the intensity of light reflected by the waste fluid received from the surgical site.

33. The method of claim 22, wherein sensing a measure related to light interacting with the waste fluid received from the surgical site includes:
transmitting a light from a laser light source through the waste fluid received from the surgical site; and
sensing a measure related to the frequency of light transmitted through the waste fluid received from the surgical site.

34. The method of claim 22, wherein sensing a measure related to light interacting with the waste fluid received from the surgical site includes:
transmitting a light from a laser light source through the waste fluid received from the surgical site; and
sensing a measure related to the frequency of light reflected by the waste fluid received from the surgical site.
